(19) United States
(12) Patent Application Publication
(10) Pub. No.: US 2008/0043905 A1
(43) Pub. Date: Feb. 21, 2008

(54) Portable Prone Stereotactic Mammography System for Biopsies, Image Guided Lumpectomies, and Radiation Treatment

(76) Inventor: Bamdad Hassanpourgol, McAllen, TX (US)

Correspondence Address: GUNN & LEE, P.C., 700 N. ST. MARY'S STREET, SUITE 1500, SAN ANTONIO, TX, 78205, US

(21) Appl. No.: 11/842,329

(22) Filed: Aug. 21, 2007

Related U.S. Application Data
(60) Provisional application No. 60/823,059, filed on Aug. 21, 2006.

(51) Int. Cl. A61B 6/02

(52) U.S. Cl. 378/37; 600/429; 600/567; 606/130

(57) ABSTRACT

A stereotactic mammography system for medical treatment and/or examination of a patient, the system comprising a portable base, a stereotactic unit connected to the portable base and positionable thereby; and a detachable operating room bed extension positionable over the stereotactic unit and having an opening for positioning a breast therethrough as the patient lies in a prone position on the bed extension. The present invention allows a physician to perform prone stereotactic breast biopsies/image guided lumpectomies at any desired location.

### CROSS-REFERENCES TO RELATED APPLICATIONS

This original, nonprovisional application claims priority to and benefit of U.S. application for provisional patent No. 60/823,059, filed Aug. 21, 2006, and which is incorporated by reference herein.

### STATEMENT REGARDING FEDERALLY SPONSORED RESEARCH OR DEVELOPMENT

Not applicable.

### BACKGROUND OF THE INVENTION

The present invention relates generally to an apparatus for performing image-guided biopsies/lumpectomies and to accommodate patients for radiation treatment. More specifically, the invention relates to a portable prone stereotactic mammography system for use in radiation treatment and medical examination.

### BRIEF SUMMARY OF THE INVENTION

The present invention comprises a stereotactic mammography system for medical treatment and/or examination of a patient. The system comprises a portable base, a stereotactic unit connected to the portable base and positionable thereby, and a detachable operating room bed extension positionable over the stereotactic unit and having an opening for positioning a breast therethrough when the patient lies in a prone position on the extension. The present invention will allow faster procedure time for patients undergoing needle localization and lumpectomies as well as avoiding undergoing general anesthesia for such exams. The current standards are too costly for the hospitals/patients and cause unnecessary pain and suffering that can be avoided by this invention.

With the mobility provided by the present invention, only one physician can perform the necessary steps involving breast biopsies/image guided lumpectomies (also includes needle localization and wire localization) in one setting if required as opposed to several physicians at different locations and times. Hence, with the utilization of the invention many patients benefit from the accuracy, faster procedure time, better cosmetics, no general anesthesia, and lower cost of stereotactic breast procedures versus the alternatives such as Wire Localization Lumpectomy (the old fashion method which consists of several steps over a long time period compared to the Image Guided Lumpectomy using the invention) and Open Surgical Lumpectomy which both are costly and lengthy processes with poor cosmetic results.

### BRIEF DESCRIPTION OF THE SEVERAL VIEWS OF THE DRAWING

The present invention, as well as further objects and features thereof, are more clearly and fully set forth in the following description of the preferred embodiment, which should be read with reference to the accompanying drawings, wherein:

FIG. 1 is a perspective view of a detached mobile prone Stereotactic mammography system for biopsies, image guided lumpectomies and radiation treatment with a motorized surgical bed, extension designed for Stereotactic mammography system for biopsies, digital detector, automated compression paddle, remote control, pullout table for surgical instruments, computer, and x-ray instrument;

FIG. 2 (A) is a side view of an extension bed;

FIG. 2 (B) is a plan view of the extension bed;

FIG. 3 is a perspective view of the workstation with the extension bed stored behind the workstation;

FIG. 4 is a side perspective view of a dedicated stereotactic table; and

FIG. 5 is an enlarged perspective view of the stereotactic unit and base.

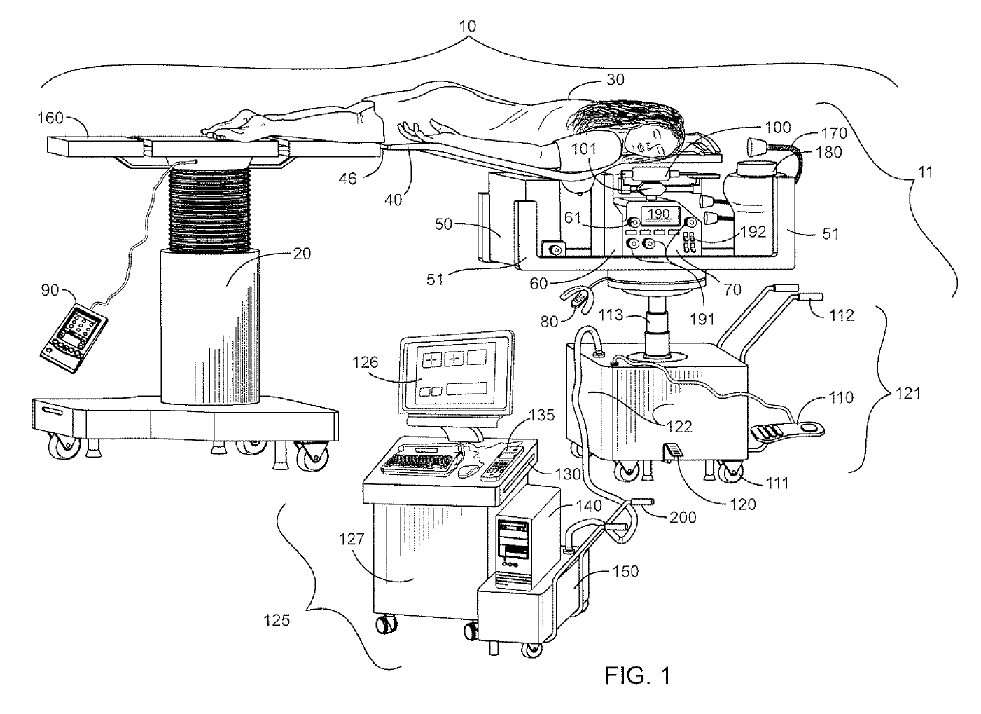

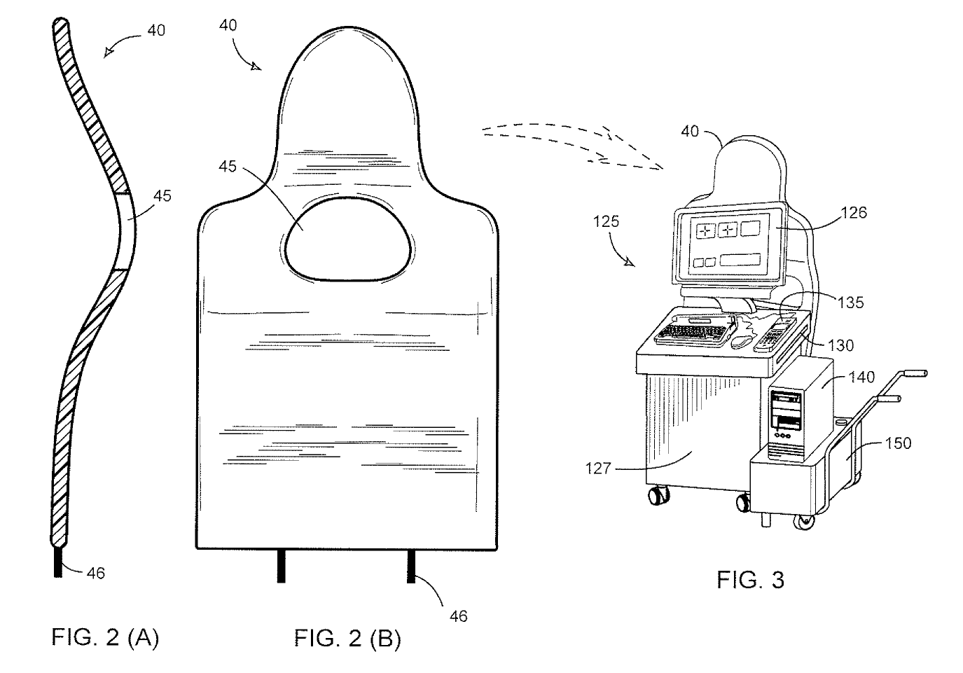

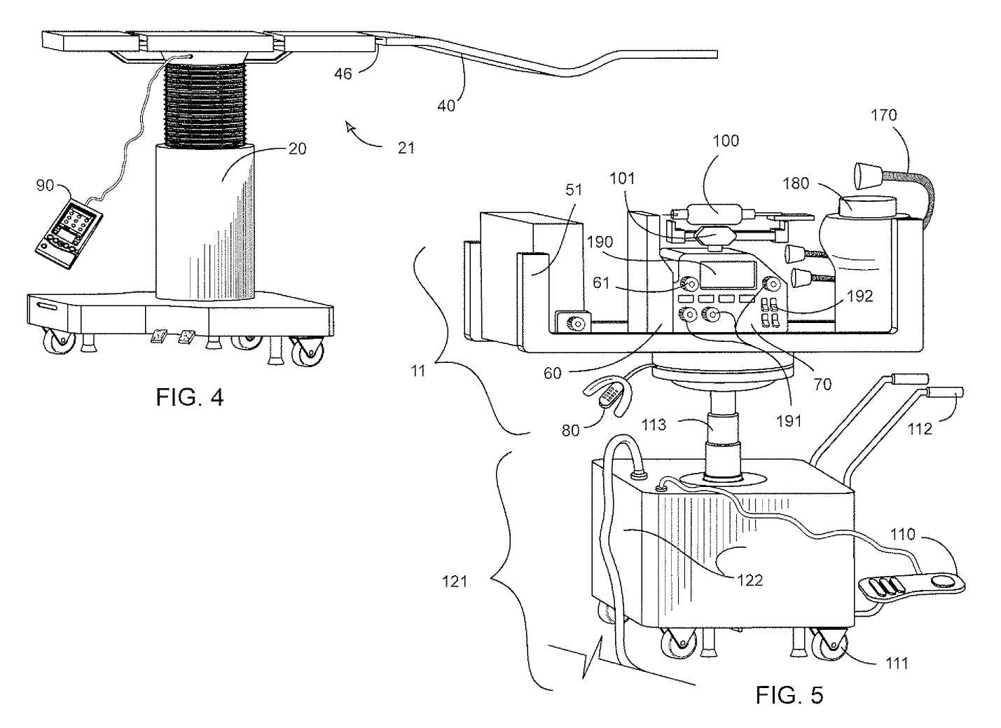

### NUMBER DESCRIPTION LIST

10 is a Portable Prone Stereotactic mammography system of the invention.
11 is a stereotactic unit.
20 is a motorized surgical bed.
21 is a dedicated stereotactic table.
30 is a patient.
40 is an extension bed designed for Stereotactic mammography.
45 is a hole for patient's breast.
46 is a prong connector.
50 is a digital detector.
51 is a stereo arm.
60 is a compression paddle.
61 is a compression paddle knob.
70 is a Stereotactic component.
80 is a control button.
90 is a remote control.
100 is a biopsy instrument.
101 is a stage.
110 is a foot pedal.
111 is a wheel.
112 is a handle.
113 is a motorized arm.
120 is a brake.
121 is a unit base.
122 is a wall panel.
125 is a mobile workstation unit.
126 is the workstation monitor.
127 is a workstation cart.
130 is a pullout table for surgical instruments.
135 is an x-ray/exposure control unit.
140 is a computer.
150 is an x-ray generator.
160 is a mattress.
170 is a light.
180 is an x-ray tube.
190 is an LCD screen.
191 are the X, Y, Z knobs.
192 are the control buttons.
200 is a handle.

### DETAILED DESCRIPTION OF THE INVENTION

A core component of this invention is a portable stereotactic unit “Base” (121) located under the stereotactic unit and stage connected with a motorized arm (113) that enables the system to move up, down, left, right, horizontal rotation on its axis, and ultimately with its wheels provides mobility to the stereotactic unit (11). This detached base unit (including wheels and motorized arm) is one of the features that makes this invention unique. The base unit's wall panels (122) are removable allowing easy access to the motor and other mechanical/electrical parts. The wall panels (122) should be made of durable materials and can be in any shape or size. Other main accessories to the this base unit are but not limited to: a) brakes (120) for stability, b) foot pedal (110) to control height adjustments, compression, and all directional movements, and c) stereotactic unit hand control buttons (80) to allow the same controls available by the foot pedal using hands which can be accessed from any side of the stereotactic unit using its rotational movement and height control for the optimum access.

A second component is a detachable Operating Room bed extension (40) which is another unique feature of this invention. The Stereotactic Prone table attachment (40) for Existing Operating Room beds allows for the patient to lie down on the table in the prone position with the effective breast hanging through the opening (hole) (45). The table is then raised to the desired height allowing the Mammographer/Physician to position the breast for X-Ray and performing the procedure. This Attachment/extension (40) can be made with Aluminum, Stainless Steel, Fiberglass, Carbon Fiber or any combination of such materials and it is connected to the standard Operating Room Bed/Surgical bed (20) using its prong connectors (46) inserted to the Operating Room bed connection holes. Another embodiment of the present invention includes a portable motorized stereotactic bed (21) that has a built in positioning hole (45) within its top surface. This alternative will make the bed/table almost exclusive for stereotactic procedures.

A third component is a stereotactic unit (11) which is designed to go under the stereotactic table allowing positioning of the breast that is hanging through the table hole (45) for X-Ray and performance of Biopsies/Image Guided Lumpectomies, and etc. The main components of this unit are: a) X-Ray tube (180) for exposure and image acquisition of the affected breast, b) X-Y-Z Stage (101) where the biopsy instruments and other devices can be mount to, c) LCD panel (190) for visualization of the coordinates and other settings, d) X-Y-Z knobs (191) for movement of the stage and device, e) control buttons (192) to adjust settings on the LCD and motorized stage movement, f) Compression Paddle (60) to position the affected breast for radiograph and procedure utilizing the manual knob (61)/foot pedal (10)/or hand control buttons, g) Digital Detector (50) which receives the radiographic image of the affected breast and converts it to the digital image sending it to the computer workstation (140) for processing/targeting, h) stereo arm (51) which connects the x-ray tube and the digital detector together allowing 15 degrees of movement in both direction in order to obtain stereotactic images, and i) motor to move the stage up/down/left/right in order to position devices to the targeted areas. Another component is the mobile workstation cart (125) which consists of a) Computer Workstation that processes and manages data, b) workstation monitor (126) which displays images and files for targeting and manipulation, c) X-Ray Generator (150) which generates power for the x-ray exposures and it is uniquely mounted on the workstation cart (127) for complete mobility of the system in use, d) pullout table tray (130) which will give an additional workspace, and e) X-Ray controls (135) that allow manipulation and adjustment of the technical factors (Kvp, mAs, and filters) in order to capture optimum X-Ray images.

In addition, though not required, it is ideal for the mobile Workstation cart (125) to be detached from the stereotactic unit (10) for ease of movement. Also lights (170) and the pullout tray (130) are recommended for ease of operation. The unique contoured shape of the OR bed attachment (40) is also preferred for patient comfort as well as maximum access to the breast lesions.

Another embodiment of the present invention (10) includes a dedicated motorized portable stereotactic bed similar to 21 with the stereotactic unit (11) and arm (113) attached to it.

This invention can be used for diagnostic breast biopsies as well as image guided needle localization and lumpectomies. In addition, after performing the image guided lumpectomy, a Brachytherapy probe can be inserted into the breast cavity irradiating the capsule (lumpectomy site) for treating the cancerous area. This method can tremendously reduce the amount of time needed to diagnose, remove, and treating the cancerous breast lesions changing the way of today's standards for breast care management.

In addition to breast biopsies and Image guided lumpectomies, this invention can accommodate the use of Internal Beam Radiation Therapy & Balloon insertion for preserving the cavity, Ultrasound for the Breast, and PET Imaging of the Breast. In the event of a suspicious lesion, a balloon can be inserted into the biopsied area protecting the cavity for possible insertion of the Internal Beam Radiation Therapy probe. In the event of cancerous findings in the specimen with negative margins, a Radiation Therapy probe can be inserted into the cavity in order to radiate and treat all the surrounding walls of the cavity for total elimination of any cancerous cells in the region of interest. If the biopsy and radiation treatment is done on the same day, the insertion of the balloon may not be necessary. A use of portable PET imaging device may be helpful prior and/or after the biopsy in order to detect any suspicious lesions and/or finding any presence of cancerous tissue in the breast. All these steps can be performed within the same day eliminating weeks and sometimes months of treatment for the patients with cancerous or suspicious findings.

The present invention is described above in terms of a preferred illustrative embodiment of a specifically described stereotactic mammography system, as well as alternative embodiments thereof. Those skilled in the art will recognize that alternative constructions of such a system can be used in carrying out the present invention. Other aspects, features, and advantages of the present invention may be obtained from a study of this disclosure and the drawings, along with the appended claims.

I claim:

1. A stereotactic mammography system for medical treatment and/or examination of a patient, said system comprising:
a portable base;
a stereotactic unit connected to said portable base and positionable thereby; and
a detachable operating room bed extension positionable over said stereotactic unit and having an opening for positioning a breast therethrough as said patient lies in a prone position thereon.
